(19) United States
(12) Patent Application Publication
(10) Pub. No.: US 2024/0033438 A1
(43) Pub. Date: Feb. 1, 2024

(54) CYLINDRICAL MEMBER AND INSTRUMENT

(71) Applicant: Enplas Corporation, Saitama (JP)

(72) Inventor: Koji NOGUCHI, Saitama (JP)

(21) Appl. No.: 18/339,288

(22) Filed: Jun. 22, 2023

(30) Foreign Application Priority Data
Jun. 24, 2022 (JP) 2022-102124

(51) Int. Cl. A61M 5/315

(52) U.S. Cl. CPC A61M 5/31513; A61M 2005/3131

(57) ABSTRACT

A cylindrical member is used for sucking a fluid into an inside of the cylindrical member and/or discharging a fluid to an outside of the cylindrical member by allowing a plunger to move inside the cylindrical member, the cylindrical member including: a first cylindrical tube inside which the plunger is to slide; and a second cylindrical tube being continuous with the first cylindrical tube and having an inner diameter larger than an inner diameter of the first cylindrical tube, the second cylindrical tube being configured to store a lubricant.

This application is entitled to the benefit of Japanese Patent Application No. 2022-102124, filed on Jun. 24, 2022, the disclosure of which including the specification, drawings and abstract is incorporated herein by reference in its entirety.

### TECHNICAL FIELD

The present invention relates to a cylindrical member and an instrument.

### BACKGROUND ART

In general, a plunger that slides with respect to an inner wall of a cylindrical member is used in order to discharge a fluid such as a gas or a liquid filling the inside of the cylindrical member to the outside or to suck a fluid into the inside of the cylindrical member (for example, see Patent Literature (hereinafter, referred to as “PTL”) 1).

PTL 1 describes a cartridge coupling mechanism for pushing out a liquid in a cartridge by sliding a plunger with respect to a cylindrical cartridge.

### CITATION LIST

### Patent Literature

PTL 1: Japanese Unexamined Patent Application Publication (Translation of PCT Application) No. 2013-517095

### SUMMARY OF INVENTION

### Technical Problem

However, in the plunger described in PTL 1, a malfunction and an imperfect shape due to wear may be caused by oil shortage. Therefore, the plunger described in PTL 1 has scope for improvement in slidability with respect to the cartridge.

In view of the above, an object of the present invention is to provide a cylindrical member allowing stable operation of a plunger. Another object of the present invention is to provide an instrument having the cylindrical member.

### Solution to Problem

[1] A cylindrical member used for sucking a fluid into an inside of the cylindrical member and/or discharging a fluid to an outside of the cylindrical member by allowing a plunger to move inside the cylindrical member, the cylindrical member including: a first cylindrical tube inside which the plunger is to slide; and a second cylindrical tube being continuous with the first cylindrical tube and having an inner diameter larger than an inner diameter of the first cylindrical tube, the second cylindrical tube being configured to store a lubricant.

[2] The cylindrical member according to [1], in which a tapered surface whose inner diameter decreases toward the first cylindrical tube is disposed on an end portion of the second cylindrical tube on the first cylindrical tube side.

[3] An instrument for sucking and/or discharging a fluid, the instrument including: a cylindrical member according to [1] or [2]; and a plunger disposed slidably inside the cylindrical member, in which a ring portion that is larger than the inner diameter of the first cylindrical tube and smaller than the inner diameter of the second cylindrical tube is disposed on the plunger.

[4] The instrument according to [3], in which a lubricant is disposed on an inner circumferential surface of the second cylindrical tube.

[5] The instrument according to [3] or [4], in which the plunger includes a plunger body, a first surface disposed on an outer circumferential surface of the plunger body and configured to make contact with an inner circumferential surface of the first cylindrical tube, the ring portion disposed on the outer circumferential surface of the plunger body, and a second surface disposed between the first surface and the ring portion.

### Advantageous Effects of Invention

According to the present invention, it is possible to provide a cylindrical member allowing stable operation of a plunger, and an instrument including the cylindrical member.

### BRIEF DESCRIPTION OF DRAWINGS

FIG. 1 is a sectional view illustrating a configuration of an instrument according to an embodiment of the present invention;

FIG. 2 is a diagram illustrating a configuration of a cylindrical member according to an embodiment of the present invention;

FIGS. 3A to 3D illustrate a configuration of a plunger according to an embodiment of the present invention;

FIGS. 4A and 4B are schematic views for explaining a using method for using the instrument;

FIGS. 5A and 5B are schematic views for explaining the using method for using the instrument;

FIG. 6 is a view for explaining angles of a tapered surface and of a side surface of a ring portion with respect to an axis; and

FIGS. 7A and 7B are schematic views for explaining the using method for using the instrument.

### DESCRIPTION OF EMBODIMENTS

An instrument according to an embodiment of the present invention is described below with reference to the accompanying drawings.

### Configuration of Instrument

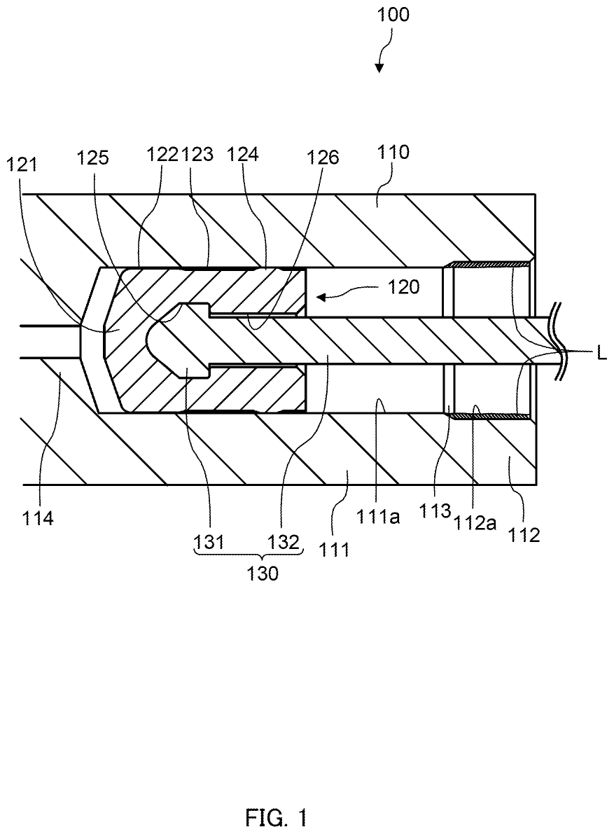

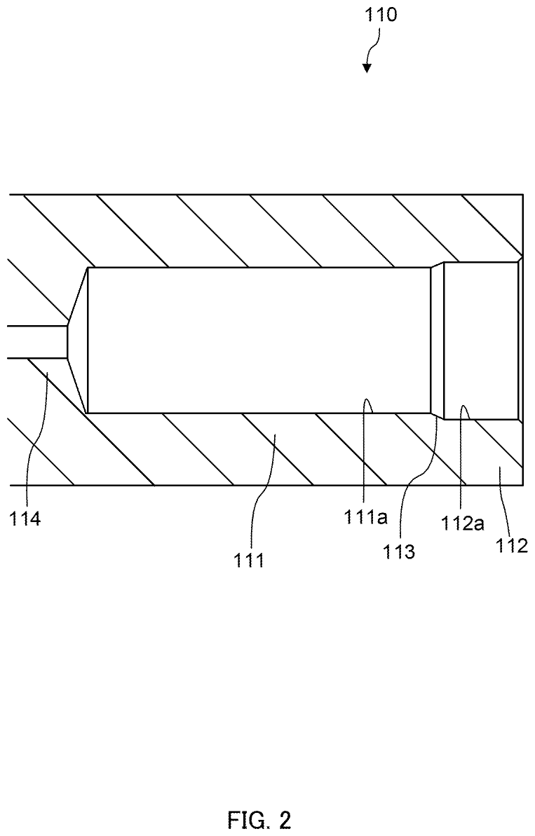

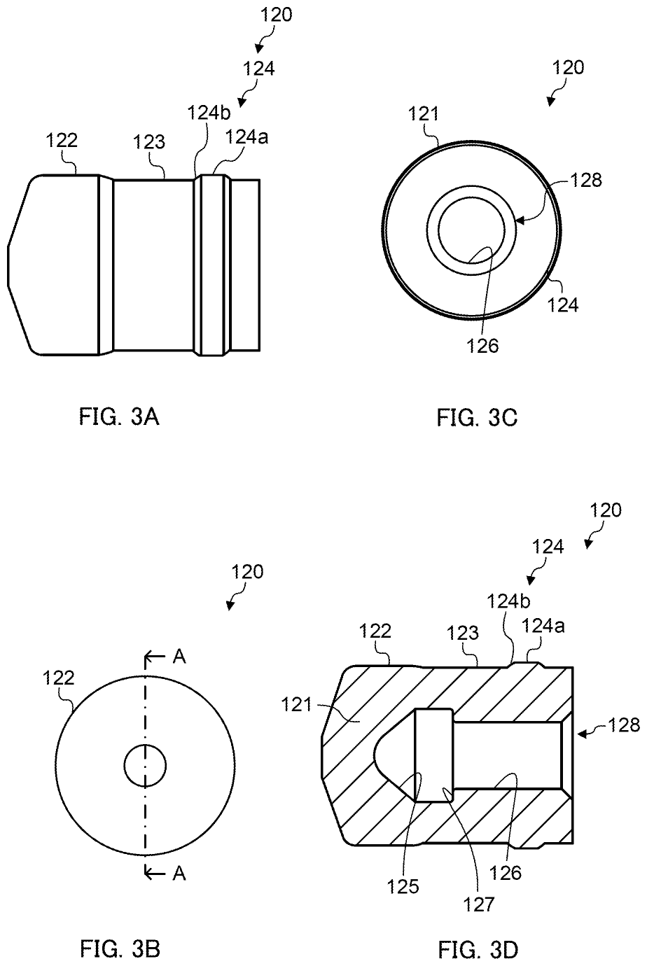

FIG. 1 is a diagram illustrating a configuration of an instrument according to an embodiment of the present invention. FIG. 2 is a sectional view illustrating a configuration of a cylindrical member according to the present embodiment. FIGS. 3A to 3D are diagrams illustrating a configuration of a plunger according to the present embodiment. FIG. 3A is a front view of the plunger, FIG. 3B is a left side view, FIG. 3C is a right side view, and FIG. 3D is a sectional view taken along line A-A illustrated in FIG. 3B.

As illustrated in FIG. 1, instrument 100 includes cylindrical member 110 and plunger 120. For example, instrument 100 is connected to a device such as a microchannel chip or a genetic test cartridge to inject or suck a fluid such as a gas or a liquid. Instrument 100 is used to suck a fluid into the inside of the instrument and discharge a fluid to the outside (e.g., to the device) by moving plunger 120 relative to cylindrical member 110. In addition, by using cylindrical member 110 as a syringe, instrument 100 can also be used as a part of an injector. In the present specification, an end portion of instrument 100 in a direction in which plunger 120 moves to discharge the inside liquid may be referred to as a “distal end,” and an end portion of instrument 100 in a direction in which plunger 120 moves to suck the liquid into the inside of the instrument may be referred to as a “proximal end.” In FIGS. 1 and 2, the left end portion is the distal end and the right end portion is the proximal end.

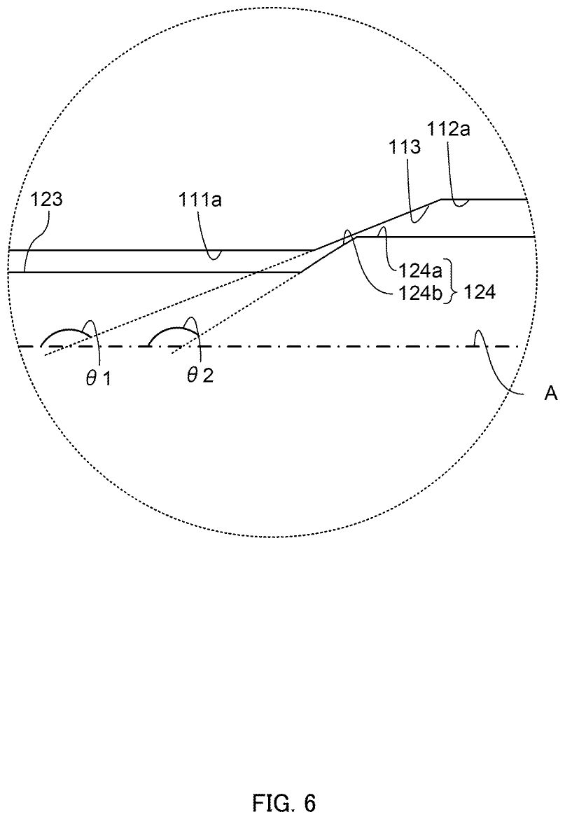

As illustrated in FIG. 2, cylindrical member 110 is a cylindrical member that is used to suck a fluid therein by movement of plunger 120 from the distal end toward the proximal end, or to discharge the inside fluid by movement of plunger 120 from the proximal end toward the distal end. Cylindrical member 110 includes first tube 111 and second tube 112. First tube 111 and second tube 112 are rotationally symmetrical with respect to axis A (see FIG. 6) along the direction in which plunger 120 moves. The material of cylindrical member 110 is not particularly limited. The material of cylindrical member 110 includes resin, glass, and metal. In the present embodiment, the material of cylindrical member 110 is polycarbonate.

First tube 111 is disposed on the distal end side of cylindrical member 110, and plunger 120 slides inside first tube 111. The cross-sectional shape of first tube 111 taken in the direction perpendicular to axis A may be circular or rectangular. In the present embodiment, the cross-sectional shape is circular. That is, the shape of inner circumferential surface 111a of first tube 111 is the shape of the side surface of a cylindrical column. Connecting tube 114 connected to the device is disposed on the distal end of first tube 111, and tapered surface 113 is continuously disposed on first tube 111. The length of first tube 111 in the direction of axis A can be appropriately set depending on the amount of the fluid to be sucked or discharged. In the present embodiment, inner circumferential surface 111a of first tube 111 as seen in a section including axis A is parallel to axis A.

Second tube 112 is disposed on the proximal end side of cylindrical member 110 and stores lubricant L. The cross-sectional shape of second tube 112 in the direction perpendicular to axis A may be circular or rectangular. In the present embodiment, the cross-sectional shape is circular. That is, the shape of a part of inner circumferential surface 112a of second tube 112 is the shape of the side surface of a cylindrical column. Second tube 112 is continuous with the proximal end of first tube 111. The inner diameter of second tube 112 is larger than the inner diameter of first tube 111. In the present embodiment, the inner diameter of second tube 112 is larger than the inner diameter of first tube 111 and is larger than the outer diameter of first surface 122 of plunger 120, which will be described later. Tapered surface 113 is disposed on a distal end portion of second tube 112. Lubricant L is disposed on inner circumferential surface 112a of second tube 112. Lubricant L disposed on the inner circumferential surface of second tube 112 is thinly spread between plunger 120 and the inner circumferential surface of first tube 111 by the operation of plunger 120. The length of second tube 112 in the direction of axis A can be set according to the storage amount of the lubricant. In the present embodiment, inner circumferential surface 112a of second tube 112 as seen in a section including axis A is parallel to axis A.

Tapered surface 113 is disposed on an end portion of second tube 112 on the first tube 111 side, and is formed to have an inner diameter decreasing toward first tube 111. The cross-sectional shape of tapered surface 113 in the direction perpendicular to axis A may be circular or rectangular. In the present embodiment, the cross-sectional shape is circular. That is, the shape of tapered surface 113 is a shape of a side surface of a truncated cone. Tapered surface 113 is disposed at a position such that first surface 122 does not come out of first tube 111 when plunger 120 is positioned at the most proximal end of cylindrical member 110.

Plunger 120 is attached to the distal end of pin 130 and is slidably disposed inside cylindrical member 110. As illustrated in FIGS. 3A to 3D, plunger 120 includes plunger body 121, first surface 122, second surface 123, and ring portion 124. In the present embodiment, plunger 120 further includes first accommodating portion 125, second accommodating portion 126, and inner stepped surface 127.

Plunger body 121 is a resin member having recess 128 that opens toward the proximal end side and in which a part of pin 130 is inserted. The resin forming plunger body 121 is, for example, an injection-moldable resin. Examples of the resin forming plunger body 121 include thermoplastic polyurethane, polyester-based thermoplastic elastomer (TPC), thermoplastic resin rubber elastomer (TPE), polyamide 612 (PA612), low-density polyethylene (LDPE), and silicone rubber. It is preferable that the Shore hardness (JIS K7215) of the resin be 95 or less. The material of plunger body 121 may include other additives in addition to the resin described above.

First surface 122 is a surface that is disposed on the outer circumferential surface of plunger body 121 and that makes a fluid-tight contact with inner circumferential surface 111a of first tube 111. First surface 122 prevents the fluid on the distal end side of first surface 122 from leaking to the proximal end side and reduces the leakage of lubricant (grease) L to the distal end side. First surface 122 may be configured to make contact with inner circumferential surface 111a of first tube 111 in a state where pin 130 is not attached. Alternatively, first surface 122 may be configured to not make contact with inner circumferential surface 111a of first tube 111 in a state where pin 130 is not attached, but to make contact with inner circumferential surface 111a of first tube 111 in a state where pin 130 is attached. In the present embodiment, first surface 122 has such a size as to make contact with inner circumferential surface 111a of first tube 111 even when pin 130 is not attached, but not to make contact with inner circumferential surface 112a of second tube 112. The shape of first surface 122 is preferably a shape complementary to inner circumferential surface 111a of first tube 111 and inner circumferential surface 112a of second tube 112. In the present embodiment, the shape of first surface 122 is the shape of the side surface of the cylindrical column.

Second surface 123 is a surface that is disposed on the outer circumferential surface of plunger body 121 continuously on the proximal end side of first surface 122 and has an outer diameter smaller than that of first surface 122. Second surface 123 is disposed between first surface 122 and ring portion 124. Second surface 123 is configured not to make contact with inner circumferential surface 111a of first tube 111 and inner circumferential surface 112a of second tube 112 in both of a state where pin 130 is not attached or a state where pin 130 is attached. The shape of second surface 123 is preferably a shape complementary to inner circumferential surface 111a of first tube 111 and inner circumferential surface 112a of second tube 112. In the present embodiment, the shape of second surface 123 is the shape of the side surface of a cylindrical column. In addition, in the present embodiment, second surface 123 as seen in a section including axis A is parallel to axis A.

In the present embodiment, first surface 122 and second surface 123 are smoothly connected to each other.

Ring portion 124 is disposed on the outer circumferential surface of plunger body 121 on the side of second surface 123 opposite to first surface 122. Ring portion 124 reduces leakage of lubricant (grease) L to the proximal end side. Ring portion 124 is preferably larger than the inner diameter of first tube 111 and smaller than the inner diameter of second tube 112. Thus, lubricant L disposed on inner circumferential surface 112a of second tube 112 can be scraped off. The shape of ring portion 124 is not limited as long as the above-described functions can be achieved. Ring portion 124 as seen in the section including axis A of plunger body 121 may have a substantially trapezoidal shape or a semi-circular shape. In the present embodiment, ring portion 124 as seen in the section including axis A of plunger body 121 has a substantially trapezoidal shape. In the present embodiment, ring portion 124 includes one top surface 124a and two side surfaces 124b (inclined surfaces). In the present embodiment, top surface 124a as seen in a section including axis A is parallel to axis A.

First accommodating portion 125 accommodates the distal end portion (head portion 131) of pin 130. The shape of first accommodating portion 125 is not particularly limited, but is preferably a shape complementary to head portion 131 of pin 130. The size of first accommodating portion 125 is preferably smaller than the size of head portion 131 of pin 130. By forming as described above, when the distal end portion (head portion 131) of pin 130 is inserted into recess 128, first accommodating portion 125 is expanded outward, and first surface 122 located outside is expanded outward.

Second accommodating portion 126 is disposed in recess 128 on the proximal end side of first accommodating portion 125, and accommodates shaft portion 132 of pin 130. Second accommodating portion 126 has an inner diameter smaller than the maximum inner diameter of first accommodating portion 125. The shape of second accommodating portion 126 is preferably complementary to shaft portion 132 of pin 130. In the present embodiment, the inner diameter of second accommodating portion 126 is smaller than the outer diameter of shaft portion 132 of pin 130.

Inner stepped surface 127 connects first accommodating portion 125 to second accommodating portion 126. In the present embodiment, inner stepped surface 127 is connected vertically to each of first accommodating portion 125 and second accommodating portion 126.

Pin 130 includes head portion 131 and shaft portion 132. Head portion 131 is disposed on the distal end of shaft portion 132. The shape of head portion 131 is not particularly limited, and is appropriately set to conform with first accommodating portion 125 of plunger 120.

Head portion 131 has a tapered surface whose outer diameter decreases from the proximal end side toward the distal end side. Head portion 131 of pin 130 is accommodated in first accommodating portion 125 of recess 128 in plunger 120. Since head portion 131 of pin 130 is engaged with inner stepped surface 127 of recess 128 in plunger 120, plunger 120 is moved to the proximal side without head portion 131 of pin 130 coming out of recess 128 of plunger 120 when pin 130 is moved to the proximal side.

Shaft portion 132 is connected to head portion 131. The shape of shaft portion 132 is not particularly limited, and is appropriately set to conform with second accommodating portion 126 in plunger 120. In the present embodiment, shaft portion 132 has the shape of a cylindrical column. The diameter of shaft portion 132 is smaller than the maximum diameter of head portion 131. Thus, the stepped surface is formed between head portion 131 and shaft portion 132. A portion of shaft portion 132 is accommodated in second accommodating portion 126 of recess 128 in plunger 120.

### Using Method for Using Instrument

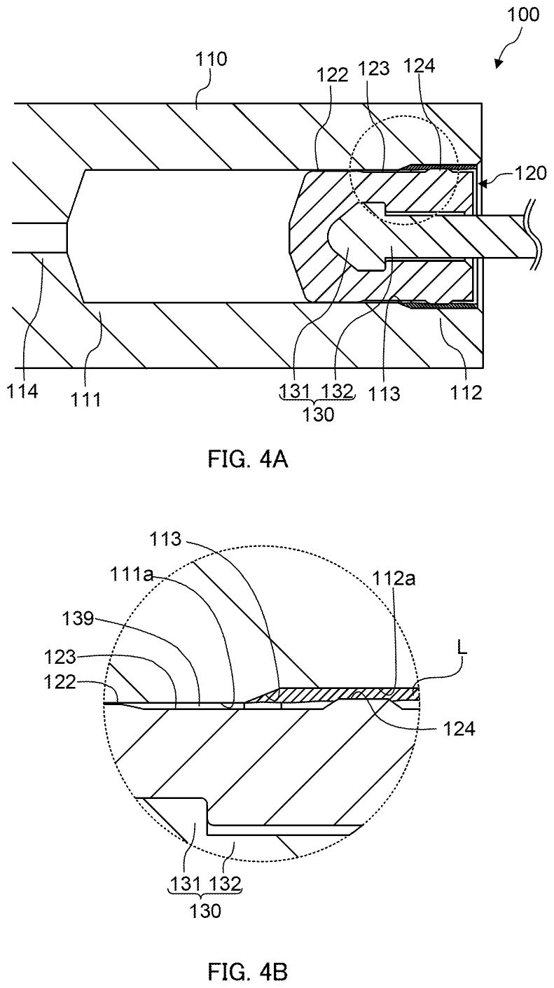

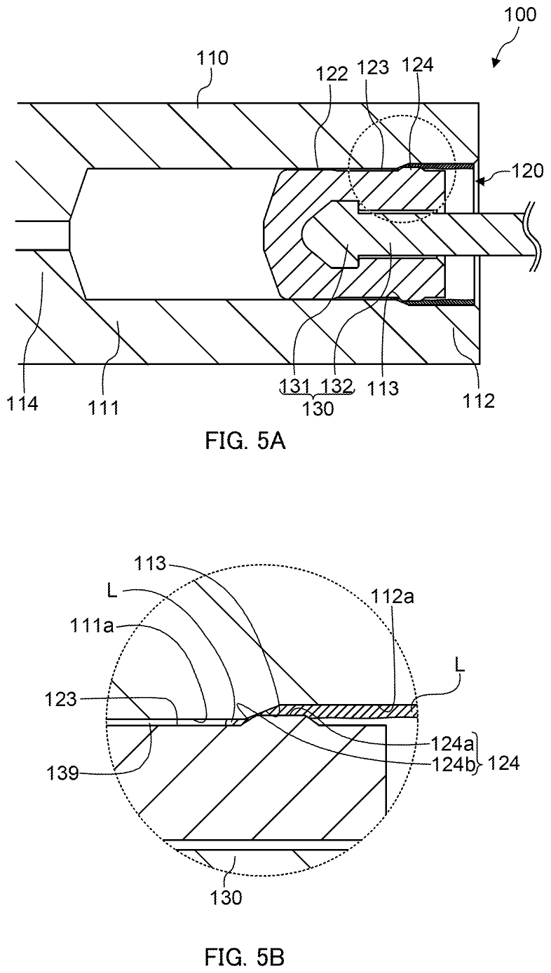

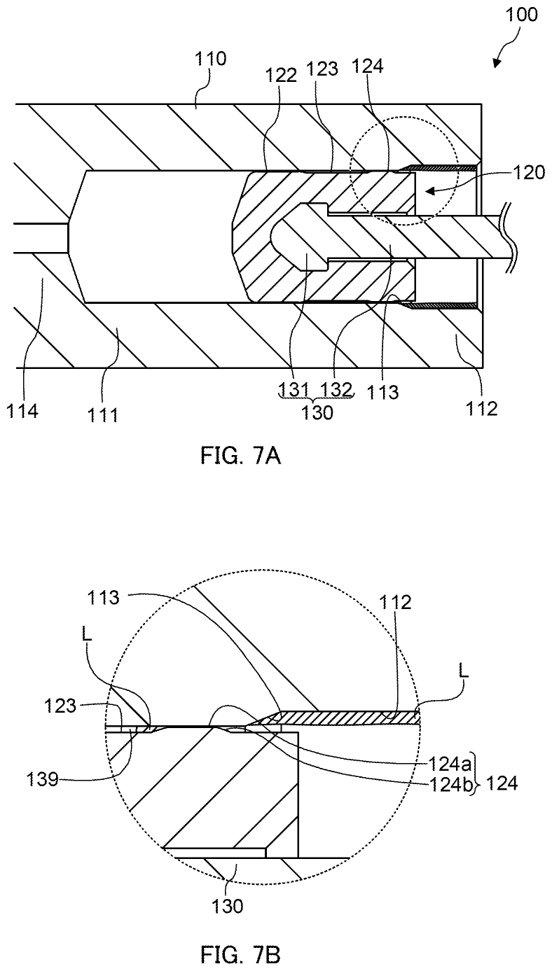

FIGS. 4A, 4B, 5A, 5B, 7A, and 7B are diagrams for explaining a using method for using instrument 100. FIG. 4A illustrates a state in which first surface 122 of plunger 120 is brought into contact with inner circumferential surface 111a of first tube 111 and ring portion 124 of plunger 120 is accommodated in second tube 112, and FIG. 4B is an enlarged view of a portion circled by a broken line illustrated in FIG. 4A. FIG. 5A is a sectional view illustrating a state in which ring portion 124 of plunger 120 is brought into contact with tapered surface 113, and FIG. 5B is an enlarged a portion circled by a broken line illustrated in FIG. 5A. FIG. 7A is a sectional view illustrating a state in which ring portion 124 of plunger 120 is brought into contact with the inner circumferential surface of first tube 111, and FIG. 7B is an enlarged view of a portion circled by a broken line illustrated in FIG. 7A. FIG. 6 is a view for explaining angles of tapered surface 113 and one of side surfaces 124b of ring portion 124 with respect to axis A.

In instrument 100 of the present embodiment, first surface 122, second surface 123, and ring portion 124 of plunger 120 are located inside first tube 111 when a fluid is sucked into cylindrical member 110 or the fluid inside cylindrical member 110 is discharged to the outside, and a part of ring portion 124 and second surface 123 moves to the inside of second tube 112 only when lubricant L is replenished. Here, an operation of replenishing lubricant L will be described.

As illustrated in FIGS. 4A and 4B, plunger 120 is moved so that ring portion 124 is located inside second tube 112. At this time, first surface 122 is in contact with inner circumferential surface 111a of first tube 111, ring portion 124 is not in contact with inner circumferential surface 112a of second tube 112, and ring portion 124 (side surfaces 124b of ring portion 124) is in contact with lubricant (grease) L.

As illustrated in FIGS. 5A and 5B, when plunger 120 is moved toward the distal end, one of side surfaces 124b of ring portion 124 comes into contact with tapered surface 113. At this time, side surface 124b of ring portion 124 scrapes off lubricant L, and lubricant (grease) L moves to a position corresponding to first tube 111. At this time, since both of side surface 124b of ring portion 124 and tapered surface 113 are inclined surfaces with respect to axis A, ring portion 124 can smoothly move from second tube 112 to first tube 111. As illustrated in FIG. 6, from the viewpoint of facilitating introduction of lubricant L into the inside of first tube 111, angle θ1 of tapered surface 113 with respect to axis A as seen in the section including axis A is preferably larger than angle θ2 of side surface 124b of ring portion 124 with respect to axis A. That is, in the present embodiment, a smaller one of the angles formed between inner circumferential surface 111a of first tube 111 and tapered surface 113 and between inner circumferential surface 112a of second tube 112 and tapered surface 113 as seen in the section including axis A is greater than a smaller one of the angles formed between second surface 123 and side surface 124b of ring portion 124 and between top surface 124a of ring portion 124 and side surface 124b of ring portion 124.

As illustrated in FIGS. 7A and 7B, when plunger 120 is moved toward the distal end, ring portion 124 makes contact with the inside of first tube 111. Accordingly, lubricant L is disposed in a space (groove 139) defined between inner circumferential surface 111a of first tube 111 and second surface 123 of plunger 120.

By moving plunger 120 inside first tube 111 in this state, lubricant L can be spread on the inner circumferential surface of cylindrical member 110 (first tube 111). Thus, the resistance between cylindrical member 110 and plunger 120 is reduced, and the operation of plunger 120 can be stably performed.

### Effects

As described above, according to the present invention, the cylindrical member includes first tube 111 and second tube 112 having an inner diameter larger than that of first tube 111. Thus, lubricant L can be replenished during use, and plunger 120 can be stably operated.

### INDUSTRIAL APPLICABILITY

The cylindrical member and the instrument of the present invention can be applied, for example, to expel or take a fluid out of or in a microchannel chip, a genetic test cartridge, or the like.

### REFERENCE SIGNS LIST

100 Instrument
110 Cylindrical member
111 First tube
111a Inner circumferential surface of first tube
112 Second tube
112a Inner circumferential surface of second tube
113 Tapered surface
114 Connecting tube
120 Plunger
121 Plunger body
122 First surface
123 Second surface
124 Ring portion
124a Top surface
124b Side surface
125 First accommodating portion
126 Second accommodating portion
127 Inner stepped surface
128 Recess
130 Pin
131 Head portion
132 Shaft portion
139 Groove
A Axis
L Lubricant (grease)

1. A cylindrical member used for sucking a fluid into an inside of the cylindrical member and/or discharging a fluid to an outside of the cylindrical member by allowing a plunger to move inside the cylindrical member, the cylindrical member comprising:
a first cylindrical tube inside which the plunger is to slide; and
a second cylindrical tube being continuous with the first cylindrical tube and having an inner diameter larger than an inner diameter of the first cylindrical tube, the second cylindrical tube being configured to store a lubricant.

2. The cylindrical member according to claim 1, wherein
a tapered surface whose inner diameter decreases toward the first cylindrical tube is disposed on an end portion of the second cylindrical tube on the first cylindrical tube side.

3. An instrument for sucking and/or discharging a fluid, the instrument comprising:
a cylindrical member according to claim 1; and
a plunger disposed slidably inside the cylindrical member, wherein
a ring portion that is larger than the inner diameter of the first cylindrical tube and smaller than the inner diameter of the second cylindrical tube is disposed on the plunger.

4. The instrument according to claim 3, wherein
a lubricant is disposed on an inner circumferential surface of the second cylindrical tube.

5. The instrument of claim 3, wherein
the plunger includes:
a plunger body,
a first surface disposed on an outer circumferential surface of the plunger body and configured to make contact with an inner circumferential surface of the first cylindrical tube,
the ring portion disposed on the outer circumferential surface of the plunger body, and
a second surface disposed between the first surface and the ring portion.

6. The instrument of claim 4, wherein
the plunger includes:
the plunger body,
a first surface disposed on an outer circumferential surface of the plunger body and configured to make contact with an inner circumferential surface of the first cylindrical tube,
the ring portion disposed on the outer circumferential surface of the plunger body, and
a second surface disposed between the first surface and the ring portion.
